(19) United States
(12) Patent Application Publication
(10) Pub. No.: US 2008/0312498 A1
(43) Pub. Date: Dec. 18, 2008

(54) Method for performing a gastro-intestianl operation and a surgical instrument for sealing an incision in the human body

(76) Inventors: Clemens Moll, Aachen (DE); Christoph Boosfeld, Koeln (DE)

Correspondence Address: ST. ONGE STEWARD JOHNSTON & REENS, LLC, 986 BEDFORD STREET, STAMFORD, CT, 06905-5619, US

(21) Appl. No.: 11/940,882

(22) Filed: Nov. 15, 2007

(30) Foreign Application Priority Data
Nov. 15, 2006 (DE) 10 2006 054 218.5

(51) Int. Cl. A61B 1/273; A61B 17/32

(52) U.S. Cl. 600/104; 606/170

(57) ABSTRACT

A method for performing a gastro-intestinal operation, which includes the steps of conducting a gastroscope through a patient's esophagus into the stomach, cutting an incision in the stomach wall or the like, conducting the distal end of the gastroscope through the incision into the abdominal area, executing operation steps in the abdominal area through the gastroscope by means of endoscopic instruments, and pulling back the gastroscope and sealing the incision by applying an occluder.

### CROSS-REFERENCE TO RELATED APPLICATIONS

The present application claims priority of German patent application No. 10 2006 054 218.5 filed on Nov. 15, 2006, the content of which is incorporated herein by reference.

### FIELD OF THE INVENTION

The invention relates to method for performing gastro-intestinal operation as set forth in claim 1, and a surgical instrument for applying a sealing device to an incision in the human body as set forth in claim 10.

### BACKGROUND OF THE INVENTION

The sealing of an incision poses challenges of varying seriousness for the surgical instrument being used, depending on the location of the incision in the human body. It is especially difficult to seal an incision that results from a gastro-intestinal operation.

In such a gastro-intestinal operation, an endoscope, in this case a gastroscope, is inserted first through the esophagus into the stomach. The gastroscope as a rule comprises several working channels through which various tools, such as cutting tools or coagulation tools, can be conveyed to the distal end of the gastroscope and used there.

In one variant of a gastro-intestinal operation, through a tool of the aforementioned type, an incision is made in the stomach wall so that the distal end of the gastroscope can be inserted through the incision into the abdominal area.

In the abdominal area the desired operation can then be undertaken by means of additional tools. Then the distal end of the gastroscope is conveyed through the incision back into the stomach.

At this point in the described operation the problem underlying the invention is posed, namely that of sealing in a simple way an incision in the human body made in the course of a gastro-intestinal operation by way of a natural bodily opening.

### SUMMARY OF THE INVENTION

The foregoing problem is solved with the method under discussion by means of the characteristics of claim 1.

It is essential, first, to recognize that an occluder can basically be used to seal a described incision. The theoretical structure of an occluder is shown, for instance, in EP 0 534 696 A1/U.S. Pat. No. 5,350,399 A.

To apply such an occluder requires a control channel and a control wire running through the control channel, to which the occluder is connected. The control wire in this case can also include several individual wires.

It is also essential to recognize that a working channel of an endoscope, in this case of a gastroscope, can be used as the control channel for the occluder. This awareness opens up the possibility of a broad new field of application for occluder seals which are known in their own right, including the field of application of the aforementioned gastro-intestinal operations.

According to an additional insight, which also assumes independent significance, a surgical instrument is resorted to in order to apply the foregoing method.

To produce this surgical instrument, an occluder is inserted into a control channel, which is a working channel of an endoscope, from the proximal end of the instrument by means of a control wire connected to it. In this way the occluder reaches the distal end of the instrument and from there can be pushed out of the control channel and applied by means of the control wire.

Insertion of the occluder from the proximal end of the instrument is advantageous if the occluder can be inserted while the distal end of the instrument is positioned, for instance, in the area of the incision that is to be sealed, that is, inside the body.

Further details, characteristics, aims, and advantages of the present invention will be further examined with reference to the illustration of a preferred embodiment.

### BRIEF DESCRIPTION OF THE DRAWINGS

FIG. 1 shows an inventive surgical instrument for applying the inventive method in a full side view.

FIG. 2 shows the distal end of the inventive surgical instrument in a side view.

FIG. 3 shows the front side of the distal end of the inventive surgical instrument.

FIG. 4 shows the proximal end of the inventive surgical instrument during insertion of the occluder.

FIG. 5 shows the distal end of the invention surgical instrument in a side view with an insert positioned in the occluder.

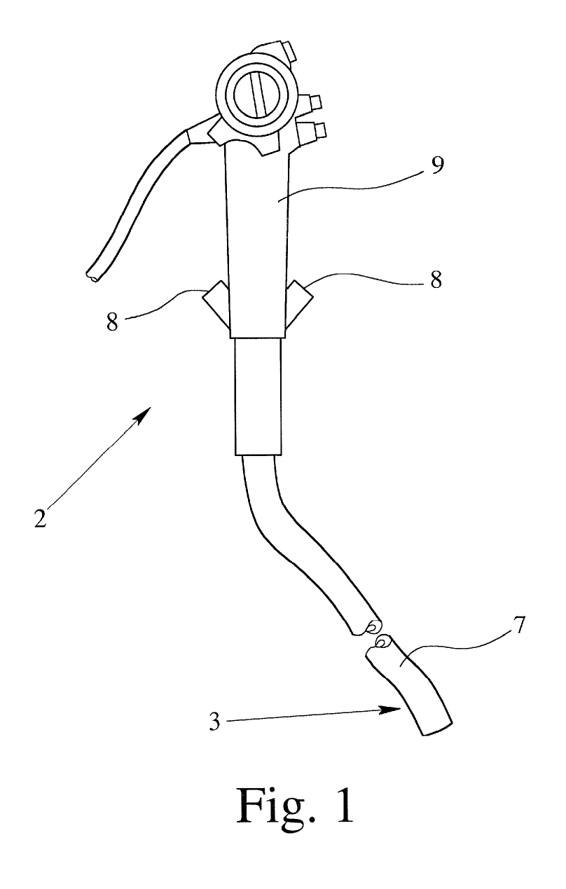

### DETAILED DESCRIPTION OF THE INVENTION

The surgical instrument shown in the illustration serves, among other uses, to apply a seal to an incision 1 in the human body by way of a natural bodily opening. The instrument comprises a control channel 4 that extends from a proximal end 2 of the instrument to a distal end 3 of the instrument. A control wire 5 runs through a control channel 4, so that an occluder 6 connected to the control wire 5 is positioned on the distal end 3 in the control channel 4.

The fundamental functioning of the occluder 6 corresponds advantageously to the functioning of the occluder known from the state of the art. The occluder 6 accordingly allows itself to be pushed out of the control channel 4 by means of the control wire 5 to be placed contiguous on the incision 1. The occluder 6 is connected to the control wire 5 preferably by a screw-in connection or the like.

The control channel 4 here is a working channel of an endoscope. Thus the inventive surgical instrument can also be designated as an endoscope and used as such.

Like every endoscope, the surgical instrument comprises an insertion tube 7, which extends from the proximal end 2 to the distal end 3. Positioned on the proximal end 2 is at least one insertion aperture 8 through which the various endoscopic tools can be inserted. Also provided on the proximal end 2 is a control body 9 for controlling the movement of the insertion tube 8.

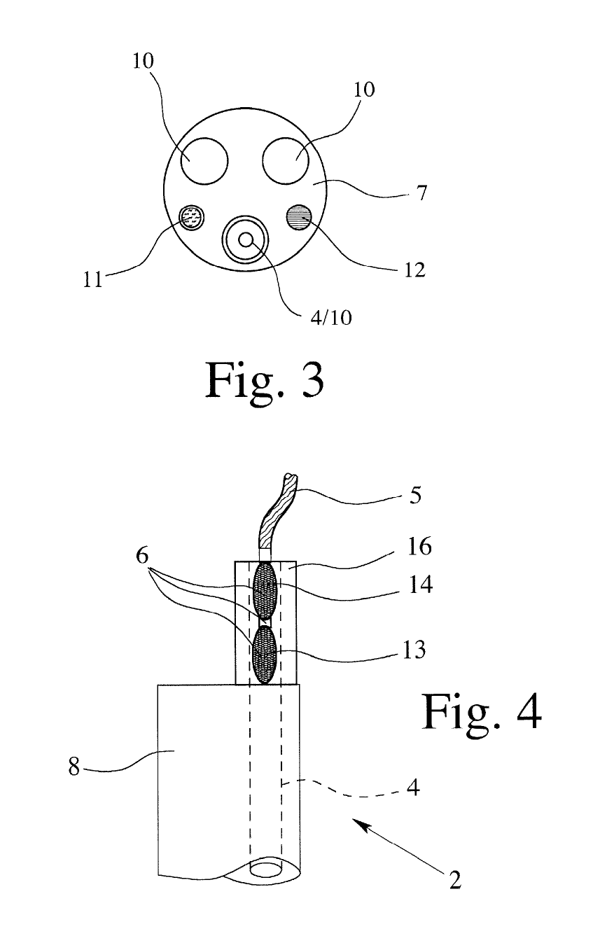

The surgical instrument, in particular the insertion tube 7, constructed in the manner of an endoscope is preferably equipped with several working channels 10, of which one channel is the aforementioned control channel 4. In the embodiment shown in FIG. 3 and thus preferred embodiment, the endoscope comprises precisely three working channels 10. It can also be seen from FIG. 3 that a lighting unit 11 and a lens system 12 are provided in the customary way on the distal end 3 of the insertion tube 7, in particular on its front side.

In an especially preferred configuration, the surgical instrument takes the form of a gastroscope with which gastro-intestinal operations can be performed. One such operation was cited in the introductory section of the description text of this document. In this case the device is preferably positioned in such a way that an incision 1 can be sealed in a stomach wall or the like through the patient's esophagus by means of the surgical instrument, said incision has previously been implemented by means of a cutting tool of the like for the passage of the gastroscope into the abdominal area.

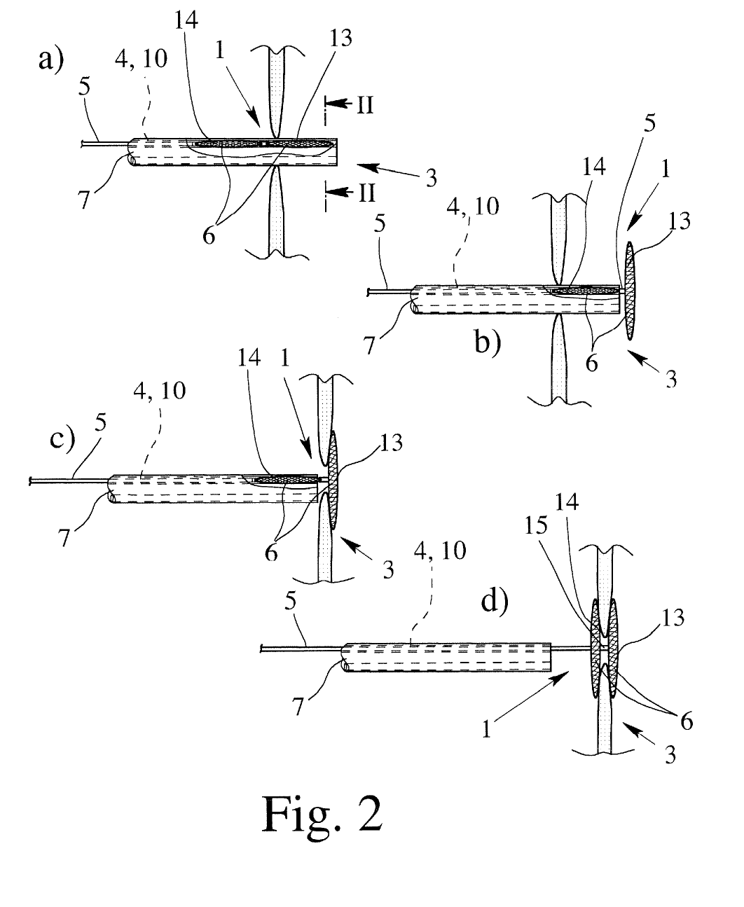

FIG. 2 shows the entire application process in three application steps. It can be seen from an overview of FIGS. 2a and 2d that the occluder 6 comprises a first collapsible and expandable screen 14 and that the two screens 13, 14 are connected with one another by a stud 15. Here the expansion of the two screens 13, 14 preferably occurs by means of a pre-tensing or a form-memory arrangement in pushing out the occluder 6 from the control channel 4 in self-actuating manner.

It can be seen from FIG. 2a that the occluder 6, before its application to the incision 1, is positioned inside the control channel 4 with both screens 13, 14 in collapsed position.

In applying the occluder 6 to the incision 1 by means of the surgical instrument, the sequence is preferably as follows.

In a first application step (FIG. 2a) the first screen 13 allows itself to be pushed out of the control channel 4 by means of the control wire 5. As shown in FIG. 2b, the first screen 13 can be expanded on the exit side of the incision 1, in FIG. 2 on the right side, while the second, still collapsed, screen 14 remains in the control channel 4.

In a second application step, the first screen 13 can be brought into contiguity with the peripheral area of the incision 1 on its exit side by means of an appropriate movement of the surgical instrument and thus of the control channel 4. The second application step can be seen in a combination of FIG. 2b with FIG. 2c.

In a third application step, the second screen 14 allows itself to be pushed out of the control channel 4 by means of the control wire 5. The second screen 14 can then be expanded on the entry side of the incision 1, in FIG. 2 on the left side of the incision 1. This is seen from a combination of FIGS. 2c and 2d. Then the control wire 5 can be unscrewed from the occluder 6 in the event of a screw connection.

During the three application steps, the movement of the distal end 3 of the surgical instrument must be controlled by the user in appropriate manner. For example, during the third application step it may become necessary to pull the gastroscope back a bit so that the second screen 14 can expand.

Other sequences in applying the occluder 6 are also possible. What is always essential, however, is the fact that the application should occur essentially by means of an appropriate control of the movement of the control wire 5 on the one hand and of the distal end 3 of the surgical instrument on the other hand.

In addition it is always true that before the first application step and preferably also during the first application step, the control channel 4 should extend through the incision 1. After the conclusion of the second application step the instrument, however, must be pulled back far enough so that the control channel 4 is now positioned before the incision 1 on the entry side of the incision 1. This is depicted in FIG. 2c.

Various variants are possible in working out the construction of the occluder 6. In a preferred configuration the occluder 6 comprises a wire mesh from which the screens 13, 14 and the stud 15 are formed. This is only hinted at in the illustration. Essentially the occluder 6 can also take the form of a one-piece structure.

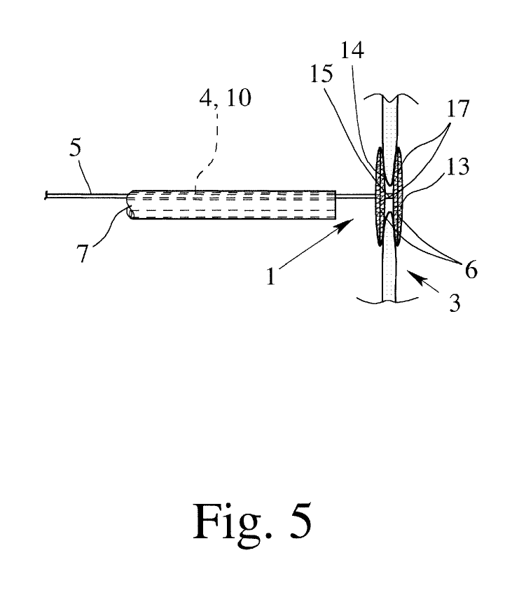

In an especially preferred configuration, an insert 17 as shown by way of example in FIG. 5 is provided in or on the occluder 6. This insert 17 can moreover be stitched together with the occluder 6. A clamping affixing device or a pressure affixing device is also possible. The insert 17 is preferably positioned in or on at least one screen 13, 14 of the occluder 6.

The above insert 17 on or in the occluder 6 can serve various functions. One function is the raising of the achievable degree of insulation of the seal of the incision 1. Another function, which can be achieved in addition or as an alternative, consists in the fact that the insert 17 promotes the regeneration of human cells that reduce the incision 1.

The configuration of the insert 17, in addition, should preferably consist of a material that is at least temporarily resistant toward the substances in the stomach, especially toward digestive juices. This means that the insert 17 in any case should “outlive” the period that is necessary to allow a sufficient regeneration of the human cells that reduce the incision 1.

The last-named function of the insert 17 can be achieved by having the insert 17 of net-like configuration so that it produces a framework for regenerating human cells that reduce the incision 1. Another possibility for achieving this function consists in the fact that the insert 17 consists of a material or is coated with a material which promotes the regeneration of human cells that reduce the incision 1. Special types of tissues such as the Surgisis® tissue of the Cook Company® can be used here.

It can also be arranged basically that the occluder 6 at least partly, but preferably completely, is configured from reabsorbable material In one variant, the first screen 13 and the second screen 14 are made of various materials that fulfill the various demands made on the two sides of the incision 1 in such a way that the occluder 6 when applied is totally reabsorbed.

Numerous other variants can be considered in producing the occluder 6. In this connection it is important to take into account that the aforementioned term “screen” is to be understood in an especially broad sense and should not be restricted to an arrangement with wire mesh. For example, screen-type arrangements of reabsorbable, tuber-type materials can be used. It is also possible that the two screens should be of different formation and/or, as explained above, should consist of different materials.

The method for producing the aforementioned surgical instrument can now be briefly summarized. It is essential here that in the control channel 4, which is a working channel 10 of an endoscope the occluder 6 should be inserted starting from the proximal end 2 of the instrument by means of the control wire 5 connected to it, so that the occluder 6 reaches the distal end 3 of the instrument and from there can be pushed out of the control channel 4 again by means of the control wire 5 to be applied to the incision 1. The advantage of this method was explained in the general discussion of the foregoing description text.

In view of the fact that the occluder 6 is preferably collapsible and expandable and is inserted into the control channel 4 in collapsed position, the problem arises of inserting the occluder 6 into the control channel 4 without damaging the sensitive occluder 6.

To solve this problem it is preferably foreseen that the occluder 6 before insertion into the control channel 4 is positioned in an insertion sleeve 16. The insertion aid 16 holds the occluder 6 in collapsed position. Before insertion of the occluder 6, the insertion aid 16 is positioned on the control channel 4 together with the occluder 6 that is positioned in the insertion aid 16. This is illustrated in FIG. 4. Finally the occluder 6 is moved out of the insertion aid 16 and inserted directly into the control channel 4.

Numerous possibilities exist for constructing the insertion aid 16. The insertion aid 16 in this case should preferably be a simple sleeve. A conventionally built occluder 6 can be easily inserted into such a sleeve. For this purpose the control wire 5 should be pulled through the sleeve until the occluder 6 connected onto the control wire 5 reaches one end of the sleeve. By further pulling on the control wire 5 the occluder 6 can finally be drawn into the sleeve. Once the occluder 6 has been absorbed completely by the sleeve, the occluder 6 has reached its “installation position.” This is illustrated in FIG. 4.

It should also be pointed, in conclusion, that the object of the invention is primarily the aforementioned method for conducting a gastro-intestinal operation in which a gastroscope is inserted into the stomach through the patient's esophagus and an incision 1 is made in a stomach wall or the like, preferably in order to conduct the distal end 3 of the gastroscope through the incision 1, and in which the incision 1 is sealed up by means of an occluder 6 after the pulling back the gastroscope. In a preferred configuration the occluder 6 is connected onto a control wire 5 and when applied onto the incision 1 through a working channel 4 of the gastroscope. All the aforementioned comments that serve to describe this method have corresponding applicability.

1. A method for performing a gastro-intestinal operation, said method comprising the following steps:
conducting a gastroscope through a patient's esophagus into the stomach,
opening an incision in the stomach wall or the like,
conducting the distal end of the gastroscope through the incision into the abdominal area,
executing steps in the operation in the abdominal area through the gastroscope by means of endoscopic instruments,
withdrawing the gastroscope and sealing up the incision by applying an occluder.

2. A method as set forth in claim 1, wherein the occluder comprises a first collapsible and expandable screen and a second collapsible and expandable screen, wherein the two screens are connected with one another by means of a stud, wherein the expansion of the two screens occurs through self-actuation by means of a pre-tensing or a shape-memory device upon pushing the occluder out of the control channel, and wherein the application of the occluder includes the following steps:
pushing the first screen out of the control channel by means of the control wire and in so doing expanding the first screen on the exit side of the incision while the second screen remains in the control channel (first application step),
moving the gastroscope in such a way that the first screen comes into contiguity with the peripheral area of the incision on its exit side (second application step),
moving the gastroscope in such a way that the second screen exits from the control channel and expands (third application step).

3. A method as set forth in claim 2, wherein the occluder, before it is applied on the incision, is positioned inside the control channel with both screens in collapsed position.

4. A method as set forth in claim 2, wherein before the first application step and preferably also during the first application step, the control channel extends through the incision and wherein after conclusion of the second application step the control channel is positioned before the incision on the entry side of the incision.

5. A method as set forth in claim 2, wherein the occluder comprises a wire mesh out of which the screens and the stud are formed.

6. A method as set forth in claim 2, wherein an insert is provided and is possibly stitched in place in or on at least one screen of the occluder.

7. A method as set forth in claim 6, wherein the insert increases the achievable degree of insulation of the seal and/or wherein the insert promotes regeneration of human cells that reduce the incision.

8. A method as set forth in claim 6, wherein the insert consists of a material that at least temporarily is resistant to the substances found in the stomach.

9. A method as set forth in claim 6, wherein the insert is of net-like configuration and provides a framework for regenerated human cells that close the incision.

10. A surgical instrument for sealing an incision in the human body through a natural bodily opening, wherein the instrument comprises a control channel extending from a proximal end of the instrument to a distal end of the instrument and a control wire running through the control channel, wherein an occluder is positioned on the distal end in the control channel, connected onto the control wire, and can be pushed out of the control channel, by means of the control wire for sealing the incision, and wherein the control channel is a working channel of an endoscope.

11. A surgical instrument as set forth in claim 10, wherein the endoscope comprises several working channels.

12. A surgical instrument as set forth in claim 11, wherein the surgical instrument is configured as a gastroscope.

13. A surgical instrument as set forth in claim 10, wherein the arrangement is made in such a way that an incision in a stomach wall or the like can be sealed through the esophagus by means of the surgical instrument, after said incision has been made by means of a cutting tool or the like as a passageway for inserting the gastroscope into the abdominal area.

14. A surgical instrument as set forth in claim 10, wherein the occluder comprises a first collapsible and expandable screen and a second collapsible and expandable screen and wherein the two screens are connected with one another by a stud.

15. A surgical instrument as set forth in claim 14, wherein the expansion of the two screens takes place by self-actuation by means of a pre-tensing or shape-memory device upon pushing the occluder out of the control channel.

16. A surgical instrument as set forth in claim 14, wherein the occluder before its application on the incision is positioned inside the control channel with both screens in the collapsed position.

17. A surgical instrument as set forth in claim 14, wherein for applying the occluder, in a first application step the first screen can be pushed out of the control channel by means of the control wire and can be expanded on the exit side of the incision while the second, collapsed screen remains in the control channel, and in a second application step the first screen can be brought into contiguity with the peripheral area of the incision on its exit side through an appropriate movement of the surgical instrument and thus of the control channel, and in a third application step the second screen can be pushed out of the control channel by means of the control wire and can be expanded on the entry side of the incision.

18. A surgical instrument as set forth in claim 17, wherein before the first application step and preferably also during the first application step, the control channel extends through the incision and wherein after conclusion of the second application step the control channel can be positioned before the incision on the entry side of the incision.

19. A surgical instrument as set forth in claim 14, wherein the occluder comprises a wire mesh from which the screens and the stud are formed.

20. A surgical instrument as set forth in claim 14, wherein an insert is provided and is possibly stitched in place in or on at least one screen of the occluder.

21. A surgical instrument as set forth in claim 20, wherein the insert increases the achievable degree of insulation of the seal and/or wherein the insert promotes the regeneration of human cells that reduce the incision.

22. A surgical instrument as set forth in claim 20, wherein the insert consists of a material that is at least temporarily resistant to the substances found in the stomach.

23. A surgical instrument as set forth in claim 20, wherein the insert is of net-like configuration and provides a framework for regenerating human cells that close the incision.
